(19) United States
(12) Patent Application Publication
(10) Pub. No.: US 2015/0351695 A1
(43) Pub. Date: Dec. 10, 2015

(54) MEDICAL SMARTPHONE

(71) Applicant: GrandiOs Technologies, LLC, Wilmington, DE (US)

(72) Inventor: John Cronin, Bonita Springs, FL (US)

(21) Appl. No.: 14/634,110

(22) Filed: Feb. 27, 2015

Related U.S. Application Data
(60) Provisional application No. 62/007,909, filed on Jun. 4, 2014.

(51) Int. Cl. A61B 5/00; A61B 5/021

(52) U.S. Cl. CPC A61B 5/6898; A61B 5/7475; A61B 5/7282; A61B 5/021; A61B 5/747; A61B 5/0022

(57) ABSTRACT

Methods and systems are presented for analyzing physiological data at an operating system of a user device in order to identify emergencies or medically significant events in real-time. In some embodiments, physiological data from a wearable physiological monitoring device may be analyzed in real-time on a user device (e.g., smartphone). In some embodiments, local and remote analyses of motion activity data feeds from data sources may be used to identify medically significant events in real-time.

### CROSS-REFERENCE TO RELATED APPLICATION

The present application claims the priority benefit of U.S. provisional application No. 62/007,909 filed Jun. 4, 2014 and entitled “Medical Smartphone,” the disclosure of which is incorporated herein by reference.

### BACKGROUND OF THE INVENTION

1. Field of the Invention

The present invention generally concerns medical data on a user device. More particularly, the present invention concerns real-time analysis of medical data on a user device.

2. Description of the Related Art

Existing medical monitoring devices monitor a user's physiological parameters, including, for example, blood pressure, pulse rate, electrocardiogram (ECG) signal, and blood oxygen saturation, in real-time. Typically, recorded physiological parameter data is stored on these existing monitoring devices and can be later accessed for processing and analysis by a computer and/or network connection.

Existing medical monitoring methods thus do not provide immediate analysis of real-time physiological data, including a severity assessment. Similarly, existing medical monitoring methods do not trigger immediate or automatic communications with third party medical providers based on a real-time assessment of recorded physiological parameter data.

There exists, therefore, a need for faster analysis of and response to real-time physiological data.

### SUMMARY OF THE CLAIMED INVENTION

Methods and systems are presented for analyzing physiological data at an operating system of a user device in order to identify emergencies or medically significant events in real-time. In some embodiments, physiological data from a wearable physiological monitoring device may be analyzed in real-time on a user device (e.g., smartphone). In some embodiments, local and remote analyses of motion activity data feeds from data sources may be used to identify medically significant events in real-time.

Various embodiments may include methods for analyzing physiological parameter data in real-time on a user device. These methods may include receiving user input including medical monitor settings, activating a medical monitor feature on the user device based on the medical monitor settings, and retrieving physiological parameter data in real-time from a physiological monitor associated with a user of the user device. The physiological parameter data may include values of the physiological parameter of the user measured in real-time and may be retrieved based on the medical monitor settings. These methods may further include analyzing the retrieved physiological parameter data in real-time to identify a medical event associated with the user. Such analysis of the received physiological parameter data may include determining that the physiological parameter data includes values that are outside of a normal range for the physiological parameter and that the medical event corresponds to a period of time in which the physiological parameter values remain outside the normal range. These methods may further include generating one or more medical notifications in response to identifying the medical event in real-time based on the medical monitor settings.

Various embodiments may further include systems for analyzing physiological parameter data in real-time on a user device. Such systems may include a user interface that receives user input that includes medical monitor settings and a processor that executes the instructions stored in the memory to activate a medical monitor feature on the user device based on the medical monitor settings. The processor may further execute instructions to retrieve physiological parameter data in real-time from a physiological monitor associated with a user of the user device. Such physiological parameter data may include values of the physiological parameter of the user measured in real-time and may be retrieved based on the medical monitor settings. The processor may further execute instructions to analyze the received physiological parameter data in real-time to identify a medical event associated with the user. Such analysis of the received physiological parameter data may include determining that the physiological parameter data includes values that are outside of a normal range for the physiological parameter and that the medical event corresponds to a period of time in which the physiological parameter values remain outside the normal range. The processor may further execute instructions to generate one or more medical notifications in response to identifying the medical event in real-time based on the medical monitor settings.

Embodiments of the present invention may further include non-transitory computer-readable storage media, having embodied thereon a program executable by a processor to perform methods for analyzing physiological parameter data in real-time on a user device as described herein.

### BRIEF DESCRIPTION OF THE DRAWINGS

FIG. 1 illustrates an exemplary network environment in which a system for analyzing physiological data in real-time on a user device may be implemented.

FIG. 2 is a diagram illustrating exemplary settings of an operating system on a user device that may be used with a system for analyzing physiological data in real-time on a user device.

FIG. 3 illustrates another network environment in which a system for analyzing physiological data in real-time on a user device may be implemented.

FIG. 4 is an exemplary diagram for generating notifications based on real-time analysis of physiological data on a user device.

FIG. 5 is an exemplary diagram for analyzing physiological data in real-time on a user device.

FIG. 6 is an exemplary diagram showing a decision matrix for analyzing physiological data in real-time on a user device.

FIG. 7 illustrates a mobile device architecture that may be utilized to implement the various features and processes described herein.

### DETAILED DESCRIPTION

Methods and systems are presented for analyzing physiological data at an operating system of a user device in order to identify emergencies or medically significant events in real-time. In some embodiments, physiological data from a wearable physiological monitoring device may be analyzed in real-time on a user device (e.g., smartphone). In some embodiments, local and remote analyses of motion activity data feeds from data sources may be used to identify medically significant events in real-time.

In some embodiments, a notification (e.g., an emergency alert or notification of a physiological status/event of the user) may be sent to one or more medical service providers (e.g., a user's general practitioner or an emergency response service). In one embodiment, the physiological data analysis system provides a real-time emergency notification function embedded in the operating system of a user device.

Access to and uses of physiological data on a user device may be customized based on user settings. Devices from which to retrieve physiological data (e.g., a wearable physiological monitor) may also be customized based on user settings. User settings, including medical monitor settings, may be inputted at the user device, stored locally on the user device, and used by the user device operating system to control accelerometer actions. In some embodiments, as permitted by medical monitor settings, a health center application on the user device may analyze physiological data to identify a medically significant event. In some embodiments, this analysis is performed in real-time by the operating system of the user device.

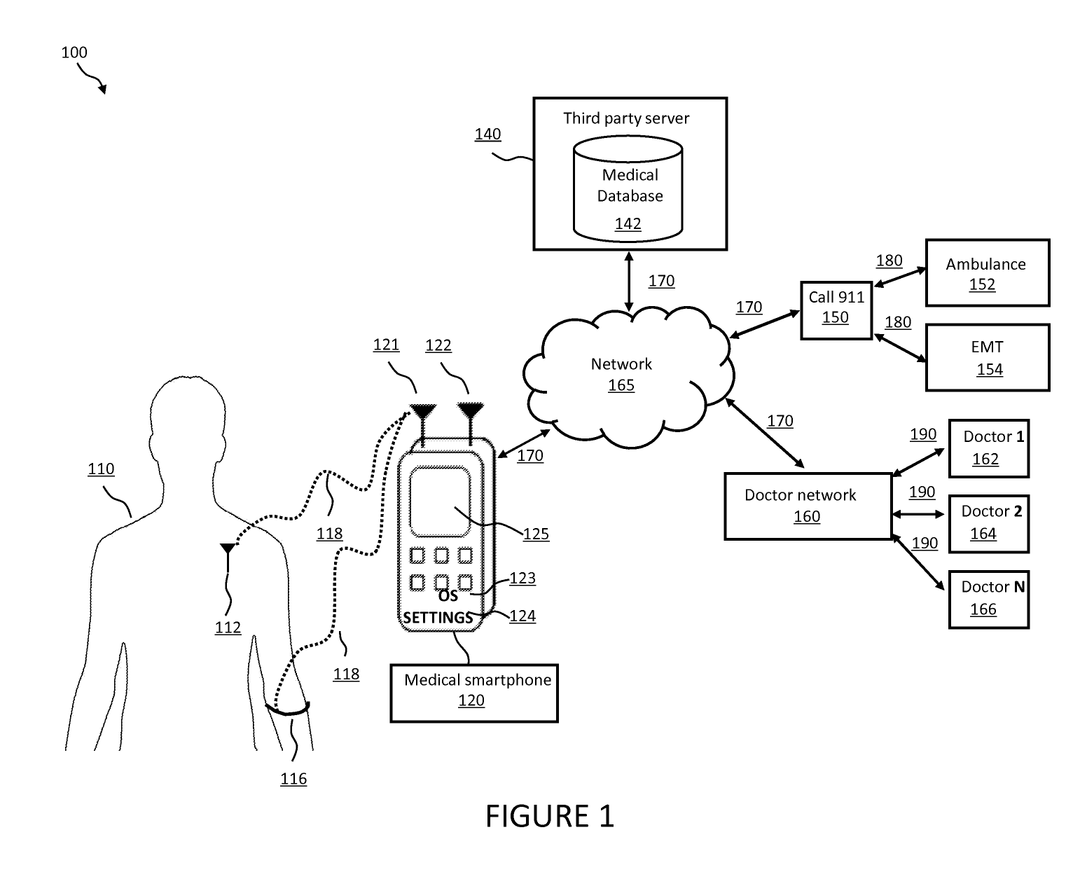

FIG. 1 illustrates an exemplary network environment 100 in which a system for analyzing physiological data on a user device may be implemented. Network environment 100 may include user device 120, network 165, network connections 170, health monitor device 120, smart watch 130, third party server 140, emergency response system 150, and receiver devices 180. Any combination of the components illustrated in network environment 100, including user device 120, network 165, network connections 170, health monitor device 120, smart watch 130, third party server 140, emergency response system 150, and receiver devices 180, and blocks, processes, or subsystems of each, and any other hardware, software, or both, for implementing the features described in the present disclosure may be collectively referred to, herein, as “the system.”

User device 120 may be any number of different electronic user devices 105, such as general purpose computers, mobile phones, smartphones, personal digital assistants (PDAs), portable computing devices (e.g., laptop, netbook, tablet), desktop computing devices, handheld computing device, or any other type of computing device capable of communicating over network 165. User device 120 may also be configured to access data from other storage media, such as memory cards or disk drives as may be appropriate in the case of downloaded services. User device 120 may include standard hardware computing components, including, for example, network and media interfaces, non-transitory computer-readable storage (memory), and processors for executing instructions that may be stored in memory.

In the illustrated embodiment, user device 120 (e.g., a smartphone) includes display 125. In some implementations, display 125 may be a touchscreen display. In some implementations, display 125 is a user interface. As shown in the illustrated embodiment, display 125 may display icons corresponding to applications (not shown). Display 125 may include any suitable soft keys. User device 120 may also include microphone 110 and on/off button 115. It will be understood that user device 120 may include other elements not shown, for example, a speaker, camera, light, or any other suitable hardware or software elements.

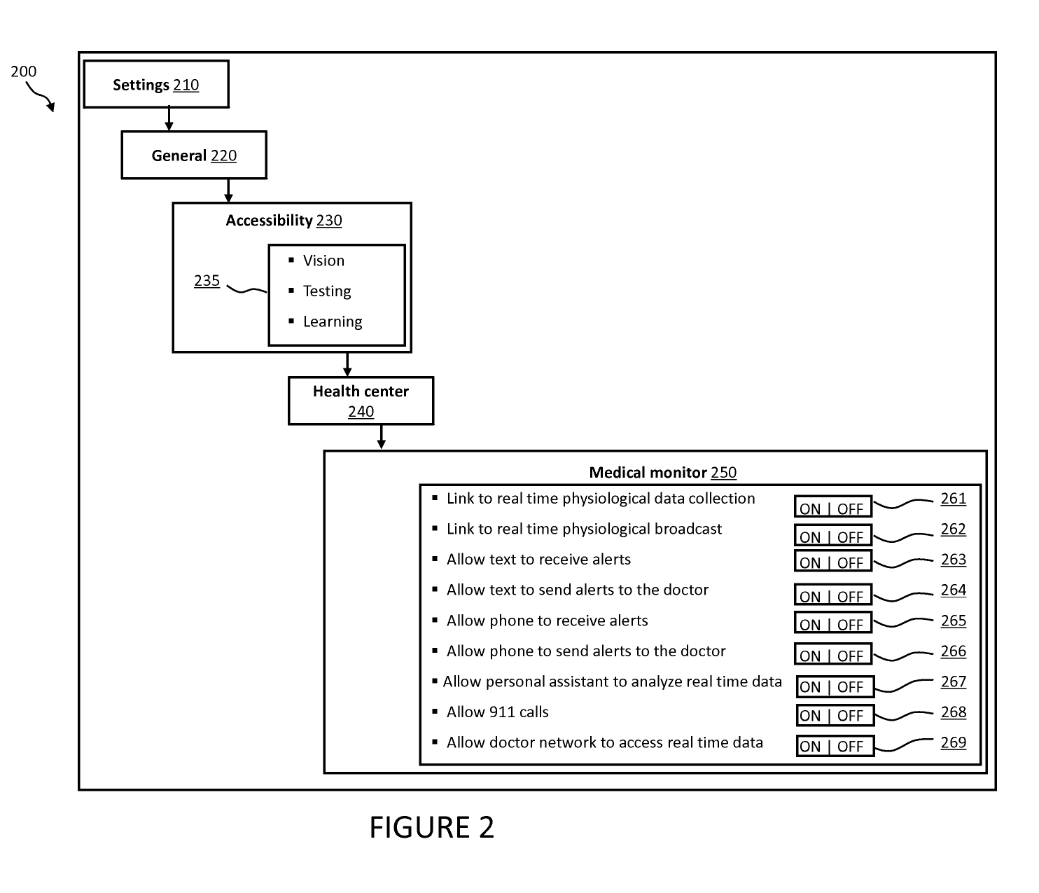

User device 120 may include operating system 123. Operating system 123 may be software that manages the use of hardware, computer programs, and applications of user device 120. Operating system 123 may be, for example, Windows, iOS, OS X, Android, UNIX, or Linux. User device 120 may additionally include settings 124, which may include configurable components of operating system 123. Settings 124 may be modifiable by a user of the user device to alter the performance of operating system 123 and other software on user device 120. In some embodiments, settings 124 may be an application on the user device 120, by which a user may select options and preferences and configure operating system functions. In an example, operating system 123 of user device 120 (e.g., an Apple device) may be iOS, and the settings 124 of user device 120 may be iOS settings. In another example, operating system 123 may be LINUX, and the settings 124 may be LINUX configuration files. In some embodiments, settings 124 may include medical monitor settings, which are modifiable by a user to alter the performance of accelerometer 125 and health center application. In some embodiments, settings 124 may be modifiable by a user to configure access to and/or sharing of physiological data with applications (not shown), health center application (not shown), third party server 140, which may include medical database 142, emergency response system 150 and doctor network 160. Settings 124 are described in detail in connection with FIG. 2. Settings 124 may also be configurable by the user to determine from which external sources physiological data may be inputted to user device 120. For example, medical monitoring devices 112 and 116 may transmit physiological data to user device 120, which may be connected to user device 120 via connection 118.

Medical monitoring devices 112 and 116 may correspond to any suitable medical monitoring devices for measuring at least one physiological parameter of a user of the user device, including, for example, blood pressure, heart rate, ECG signal parameters, blood oxygen saturation, or any other suitable physiological parameter. In the illustrated embodiment, medical monitoring devices 112 and 116 are wearable physiological monitors. In some embodiments, environment 100 may include any suitable number of medical monitoring devices 112 and 116 for inputting physiological data to user device 120.

User device 120 may include any suitable software or applications. In some embodiments, personal assistant software (not shown) runs on user device 120. The personal assistant may be software capable of performing tasks for a user based on, for example, user input, location awareness (e.g., using GPS 155), user settings 124, locally stored information and information accessible over a network (e.g., network 165) from a personal assistant server (not shown), third party server 140, applications (not shown), a social network (not shown), emergency response system 150, and medical monitoring devices 112 and 116. Existing, exemplary, personal assistants include, for example, SIRI™ services (for Apple devices), GOOGLE NOW™ services (for Google Android devices), S VOICE™ (for Samsung devices), and VOICE MATE™ services, (for LG Electronics devices). It will be understood that the examples of existing intelligent personal assistants described herein are merely exemplary, and the system of the present disclosure may be implemented using any suitable hardware and/or software.

In some embodiments, personal assistant software (not shown) is a personal assistant application running on user device 120. Personal assistant software may, for example, send messages, make telephone calls (e.g., emergency calls to specified contacts), set reminders, make calendar appointments, retrieve data locally or remotely (e.g., locally from a wearable medical monitor), analyze retrieved data, perform internet searches, provide emergency notifications, generate audio or visual output at a speaker or interface of the user device, or perform any other suitable actions in response to user input. In some embodiments, depressing an electromechanical button (e.g., on/off button) may activate the personal assistant. In some embodiments, actuating a personal assistant soft key may turn the personal assistant ON or OFF. In some embodiments, a personal assistant on user device 120 may be used to collect and analyze physiological data, to manage the input and output of physiological data, or to provide any other physiological data functionality in accordance with medical monitor settings.

Applications (not shown) are software blocks on user device 120, which may be downloaded from remote servers. Applications (not shown) may provide additional functions for user device 120. For example, applications (not shown) may be any suitable applications downloaded from, for example, Apple Inc.'s APP STORE® (for Apple devices), GOOGLE PLAY® (for Google Android devices), or any other suitable database or server. In some embodiments, applications 140 may be software, firmware, or hardware that is integrated into the user device 120. In some embodiments, a health center application may be used to retrieve and analyze physiological data, as well as to generate notifications based on the analysis of the physiological data and to manage user settings relating to the physiological data (e.g., medical monitor settings 250 described in connection with FIG. 2 below).

User device 120 may also include a local database (not shown), which may be used to store medical monitor settings and physiological data input and outputs. Local database 145 may also store physiological data retrieved from external accelerometer-enabled devices, including, for example, smart watch 130 and health monitoring device 120.

Antennas 121 and 122 are a component of user device 120. In some embodiments, user device 120 may use antennas 121 and 122 to send and receive information wirelessly. For example, antennas 121 and 122 may be cellular data antennas, Wi-Fi antennas, or BLUETOOTH® antennas. In some embodiments, antennas 121 and 122 are implemented as a single antenna. In the illustrated embodiment, antennas 121 and 122 are used to establish a BLUETOOTH connection with smart watch 130 and health monitoring device 120 and to establish a network connection with third party server 140, emergency response system 150, and receiver devices 180 over network 165.

Network connections 170 may include any suitable wired or wireless transmission mediums or channels through which data may be communicated. In the illustrated embodiment, network connections 170 may communicate data between user device 120, network 165, third party server 140, emergency response system 150, and doctor network 160. Network connections 170 may include, for example, a computer networking cable, an Ethernet cable, a cellular communications network, an Internet data trunk (e.g., single transmission channel), a wireless local area network, a wide area network, or a telecommunications network (e.g., 4G wireless network).

Network 165 may include the Internet, a system of interconnected computer networks that use a standard protocol, a dispersed network of computers and servers, a local network, a public or private intranet, any other coupled computing systems, or any combination thereof. In some embodiments, network 165 may be a cloud, which is a network of remote servers hosted on the Internet and used to store, manage, and process data in place of local servers or personal computers. User device 120 may be coupled to network 165 though any suitable wired or wireless connection. In some embodiments, user device 120 may be coupled to network 165 via network connections 170.

Network 165 may allow for communication between the user device 120, third party server 140, emergency response system 150, and receiver devices 180 via various communication paths or channels. Such paths or channels may include any type of data communication link known in the art, including TCP/IP connections and Internet connections via Wi-Fi, BLUETOOTH, a Universal Mobile Telecommunications System (UMTS) network, or any other suitable data communication link. In that regard, network 165 may be a local area network (LAN), which may be communicatively coupled to a wide area network (WAN) such as the Internet. The Internet is a broad network of interconnected computers and servers allowing for the transmission and exchange of Internet Protocol (IP) data between users connected through a network service provider. Examples of network service providers are the public switched telephone network, a cable service provider, a provider of digital subscriber line (DSL) services, or a satellite service provider. Network 165 allows for communication between any of the various components of network environment 100.

External medical monitoring devices may include any suitable remote devices for generating physiological data about the user that may be input into user device 120. In the illustrated embodiment, medical monitors 112 and 116 are shown as exemplary external medical monitoring devices. In the illustrated embodiment, smart watch 130 may include accelerometer 132, time display 134, and antenna 136. It will be understood that smart watch 130 may include any other suitable components. In the illustrated embodiment, health monitoring device 120 may include various monitoring device functions 124, accelerometer 122, and antenna 126. In some embodiments, medical monitoring devices 112 and 116 may be connected to user device 120 via connections 118.

Connections 118 may include any suitable wired or wireless transmission mediums or channels through which data may be communicated. In the illustrated embodiment, connections 118 may communicate data between user device 120, medical monitoring devices 112 and 116, and any other suitable external medical monitoring devices (not shown). Connections 118 may include, for example, a computer networking cable, an Ethernet cable, a cellular communications network, an Internet data trunk (e.g., single transmission channel), a wireless local area network, a wide area network, or a telecommunications network (e.g., 4G wireless network). In some embodiments, medical monitoring devices 112 and 116 may transmit physiological data, collected at each respective device, over connections 118 to user device 120, where it may be analyzed in real-time to immediately identify medically significant events.

Social network (not shown) may be any suitable networking platform on which a user may share data or post information. The social network may be coupled to user device 120 and network 165 via network connections 170. In some embodiments, the social network may receive user emergency event data in accordance with settings 124, and if permitted by settings 124, the social network may share the user emergency event data with the user's friends, family, connections, or a group of other users defined by the user at settings 124 of the user device or in social network settings.

Third party server 140 may retrieve physiological data outputted by user device 120 over network 165. Third party server 140 may be coupled to network 165 and user device 120 by network connections 170. In some embodiments, third party server 140 may include medical database 142 for storing physiological data outputted by user device 120 and physiological data outputted by a plurality of other user devices (not shown). In some embodiments, medical database 142 may also store user settings (e.g., settings 124) received at third party server 140 for sharing the stored physiological data in accordance with the user settings. In some embodiments, as permitted by settings 124, physiological data may be transmitted by operating system 123 of user device 120 to third party applications stored in the third party database on third party server 140. In some embodiments, a plurality of other users may also be connected to third party server 140, which manages sharing of physiological data among the plurality of users based on respective user settings, which may be stored in medical database 142. In some embodiments, a user of user device 120 may link to a second user on a separate user device in settings 124, and the user's physiological data may be shared with the linked second user via third party server 140 and third party server.

Emergency response system 150 may be any suitable emergency responder system, public or private, accessible over network 165 by way of network connections 170. In some embodiments, emergency response system 150 may be a private emergency assistance program, available to enrolled members, including for example, OnStar®, or any other suitable emergency assistance system. In some embodiments, emergency response system 150 may include a public assistance program, including, for example, 9-1-1 emergency services, or any other suitable public emergency assistance program. In some embodiments, emergency response system 150 may receive communications from user device 120 over network 165 and network connections 170 in order to determine the nature and severity of the emergency services required. In some embodiments, emergency response system 150 may dispatch assistance, for example, medical assistance or roadside assistance for automobile incidents, based on received communications from user device 120. In some embodiments, emergency response system 150 may be associated with public services, including, for example, a fire or police department, and help rendered may be from one or more of the associated public services. In some embodiments, establishing a communication with an emergency response system 150 may automatically send global positioning system (e.g., GPS on user device 120) data to emergency response system 150 for location of the user device and potential emergency situation. It will be understood that emergency response system 150 may be any suitable help dispatch system provided by any suitable purveyor of help services.

Third party server 140 and emergency response system 150 may include any type of server or other computing device as is known in the art, including standard hardware computing components such as network and media interfaces, non-transitory computer-readable storage (memory), and processors for executing instructions or accessing information that may be stored in memory. The functionalities of multiple servers may be integrated into a single server. Alternatively, different functionalities may be allocated among multiple servers, which may be located remotely from each other and communicate over the cloud. Any of the aforementioned servers (or an integrated server) may take on certain client-side, cache, or proxy server characteristics. These characteristics may depend on the particular network placement of the server or certain configurations of the server.

Doctor network 160 may include any suitable number of doctors 162, 164, and 166 (i.e., shown as doctors 1, 2, . . . n) that may receive data from and send data to user device 120 over doctor network 160, network 165, network connections 170, and doctor network connections 190. In some embodiments, a user may specify which doctors 162, 164, and 166, in a doctor network 160, should receive medical monitor notifications in user settings 124. Doctors 162, 164, and 166 may be specified emergency contacts in user settings 124, and the system may send an emergency notification to doctor network 150, which may then communicate the emergency notification to any of doctors 162, 164, and 166 according to the specified emergency contacts.

FIG. 2 is a diagram illustrating exemplary settings 200 of an operating system on a user device that may be used with a system for analyzing physiological data on a user device. In some embodiments, settings 200 may be displayed on a user interface of user device 120 of FIG. 1. In some embodiments, settings 200 may correspond to settings 124 of user device 120 of FIG. 1. Settings 200 may, for example, provide a mechanism by which a user may alter the functions of an operating system of a user device by implementing changes to settings. Settings 200 may facilitate user interaction with a user device.

Settings 200 may include settings menu 210. Settings menu 210 may include user-editable features for customizing the functionality of an operating system or user device according to user preferences. In some implementations, settings 124 of user device 120 of FIG. 1 are modifiable by a user to alter the performance of operating system 123. In some implementations, settings 124 of user device 120 of FIG. 1 are modifiable to alter the performance of an accelerometer application on user device 120. In some embodiments, settings 200 may be modified by the user interacting with options or commands in a respective settings menu 210. Settings menu 210 may include any number of user-selectable options or commands. Settings menu 210 may include any suitable number of standard operating system or user device settings, for example, general settings 220, including accessibility settings 230, as shown in FIG. 2. General settings 220 are exemplary interface elements that, when selected by a user, may, for example, redirect the user to a respective new page, window, or dialogue box.

In some embodiments, settings menu 210 includes a list of user-selectable options or settings presented in a hierarchical order. For example, accessibility settings 230, including vision, texting, and learning settings 235, may be sub-settings under general settings 220. Accessibility settings 230 may additionally include health center settings 240, which is shown as selected (e.g., underlined) in FIG. 2, and the selection of health center settings 240 may reveal a sub-menu of medical monitor settings 250. Medical monitor settings 250 may include exemplary settings 261-269 that, when selected by a user, may, for example, redirect the user to a respective new page, window, or dialogue box. In another example, when selected, any of the interface elements may expand to reveal sub-options, sub-commands, or any other suitable settings display elements.

In some embodiments, medical monitor settings 250 may include user-editable features for customizing the functionality of a health center application running on a user device. In some embodiments, medical monitor settings 250 may be used to customize the functionality of an operating system with respect to analysis of retrieved physiological data on a user device (e.g., user device 120 of FIG. 1). As illustrated in FIG. 2, medical monitor settings 250 may include a mechanism for selection and de-selection of settings. In the shown embodiment, on/off selection buttons are illustrative examples of mechanisms for selection and de-selection of automatic personal assistance settings. In some embodiments, selection and de-selection in settings menu 210 are binary selections.

In some embodiments, medical monitor settings 250 may include any suitable number of selectable medical monitor settings 250, which, in the illustrated embodiment, are depicted as including sub-settings 261-269, as shown in FIG. 2.

In the illustrated embodiment, exemplary medical monitor settings 261-269 are shown. Medical monitor settings 250 may be used to allow or disallow access to and analysis of physiological data. Medical monitor settings 250 may also be used to allow or disallow notifications to be made based on the real-time physiological data analysis. In some embodiments, automatic medical monitor settings 250 may be used to configure medical monitoring features based on user preferences.

Link to real-time physiological data collection 261 allows a user to switch on and off retrieval of physiological data from a remote physiological sensor, by the user device (e.g., accelerometer software 125 of FIG. 1) on or off. Link to real-time physiological data broadcast 262 allows a user to switch on and off broadcasting of physiological data (e.g., saving to a remote location, including, for example, a cloud server or third party server 160 of FIG. 1). In some embodiments, medical monitor settings may be used to allow or disallow local applications or external/third party servers access to physiological data. If the health center application is turned on, which it is in the illustrated embodiment, the user may then configure the sharing of physiological data. Using allow doctor network to access real-time data setting 269, a user may allow or disallow transmission of retrieved physiological data and analyses of the physiological data, including medical or emergency notifications, with a doctor or other medical provider network (e.g., doctor network 160 of FIG. 1). Using allow personal assistant to analyze real-time data setting 267, a user may allow or disallow a personal assistant of the user device access to retrieved physiological data and permission to analyze retrieved physiological data.

Other exemplary medical monitor settings may be used to configure notification functions of a medical monitor feature or health center application on the user device. Using allow emergency responder call setting 268, a user may switch on or off calls to an emergency responder system, for example 9-1-1 emergency services (e.g., emergency response system 150 of FIG. 1). Using allow text to receive alerts setting 263, a user may allow or disallow a text notification generated based on an analysis of physiological data to be sent via text message to the user device when the system detects an emergency or medically-significant event based on the physiological data. For example, if setting 263 is switched “ON,” the user device or a remote emergency response system (e.g., emergency response system 150 of FIG. 1) or a doctor network/doctor (e.g., doctor network 160 or doctors 162, 164, and 166 of FIG. 1) may be allowed to text the user device regarding physiological data and/or medical notifications. Using allow phone to receive alerts setting 265, the user may allow of disallow the notifications described above in reference to setting 263 to be made to the user device/ smartphone (e.g., user device 120 of FIG. 1). Using allow text to send alerts to doctor setting 264, a user may allow or disallow a phone notification generated based on an analysis of physiological data to be sent by phone (e.g., phone call) to a doctor network or particular doctor (e.g., doctor network 160 or doctors 162, 164, and 166 of FIG. 1) when the system detects an emergency or medically-significant event based on the physiological data. Using allow phone to send alerts to doctor setting 266, a user may allow or disallow the notifications described above in reference to setting 264 to be sent via phone to a doctor network or particular doctor. For example, the user may specify in medical monitor settings 250 the phone number and physician name or doctor contact information to which notifications may be sent.

It will be understood that the illustrated medical monitor settings are merely exemplary and not provided by way of limitation. Any suitable settings for configuring access to, retrieval of, and analysis of physiological data on the smart phone may be used. For example, settings may also be used to set which types of outputs of a user device may be used in providing a notification to a third party (e.g., family, friend, or other specified person) based on an analysis of the physiological data (e.g., email, text, phone call, or personal assistant command).

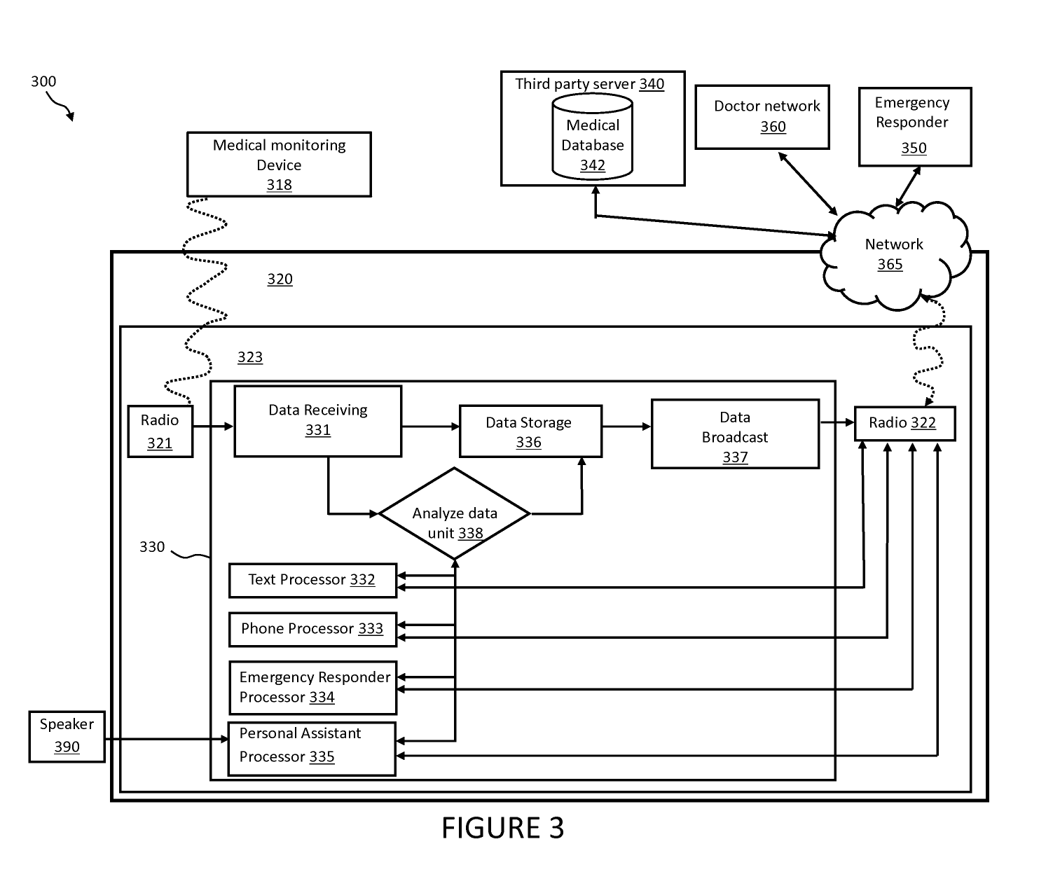

FIG. 3 illustrates another network environment 300 in which a system for analyzing physiological data in real-time on a user device may be implemented. In the illustrated embodiment, network environment 300 includes user device 320 (e.g., smartphone), which includes operating system 323 and radio 321, which is connected to medical monitoring device 318. User device 320 is also shown as including radio 322, which is connected to network 365, which is in turn connected to third party server 340, including medical database 342, doctor network 360, and emergency responder 350.

In some embodiments, user device 320, operating system 323, radio 321, and medical monitoring device 318 may correspond to respective user device 120, operating system 123, antenna 121, and medical monitoring devices 112 and/or 116 of FIG. 1. In some embodiments, radio 321 is a transmitter and receiver, including an antenna, for example, antenna 121 of FIG. 1, which sends and receives Bluetooth or other short-range communications to and from medical monitoring device 318. In some embodiments, radio 321 may be a Bluetooth radio device, near field communications device, or other suitable short-range communications device.

In some embodiments, radio 322, network 365, third party server 340, medical database 342, doctor network 360, and emergency responder 350 may correspond to respective antenna 122, network 165, third party server 140, medical database 142, doctor network 160, and emergency response system 150 of FIG. 1. In some embodiments, radio 322 is a transmitter and receiver, including a wireless antenna, for example antenna 122 of FIG. 1, which sends and receives cellular or other long- range communication to and from nearby networked locations, including, for example, cellular towers or wireless hot spots.

Health center application 330 may be a software block running on user device 320, which may be downloaded from a remote server. In some embodiments, health center application 350 analyzes physiological data from medical monitoring device 318 to provide notifications to user device 320, third party server 340, emergency responder 350, and doctor network 360. In some embodiments, health center application 330 is a software module within operating system 323 which inputs, analyzes, and outputs real-time medical monitoring data (e.g., physiological data). In the illustrated embodiment, health center application 330 includes data receiving module 331, data storage module 336, data broadcast module 337, data analysis module 338, text processor 332, phone processor 333, emergency responder processor 334 and personal assistant processor 335.

In some embodiments, health center application 330 may provide an interface for display of user settings (e.g., medical monitor settings 250 of FIG. 2) to a user of user device 120. In particular, a user may use health center application to set and view medical monitor settings (described below in connection with FIG. 2), which may be used to provide physiological data to third party server 340, emergency responder 350, and doctor network 360. Medical monitor settings may also be set in health center application 330 to specify from which external medical monitoring devices (e.g., medical monitoring device 318) physiological data may be retrieved.

Data receiving module 331 inputs real-time medical monitoring data, which may include physiological data (e.g., physiological parameter measurements) from medical monitoring device 318 and any other suitable physiological monitor. In some embodiments, data receiving module 331 includes an analog-to-digital converter for converting received analog data to a digital data signal. In some embodiments, data receiving module 331 may separate data signals received from different medical monitors or associated with different physiological parameters into appropriate data streams. For example, data signals associated with the appropriate measurements may be placed into a blood pressure data stream, pulse rate data stream, or skin conductivity data stream. After data receiving module 331 has sorted the retrieved physiological data signals into separate streams, the data is passed to both data storage module 336 and data analysis module 338.

Data storage module 336 may be any suitable memory or storage database of user device 320 for storing real-time physiological data received from medical monitoring device 318 or other medical monitoring data analysis. In some embodiments, data storage module 336 may store data based on user preferences (e.g., medical monitoring settings 250 of FIG. 2). In some embodiments, data storage module 336 saves medical monitoring data to a local memory on user device 320 in accordance with user settings. In some embodiments, data storage module 336 sends the data to data broadcast module 337.

Data broadcast module 337 is a software module that interfaces between user device 320 and network 365 to transmit and receive medical monitoring data to and from third party database 340, emergency responder 350, and/or doctor network 360. In some embodiments, data broadcast module 337 receives data from data storage module 336 for transmission to one or more of third party database 340, emergency responder 350, and doctor network 360. In some embodiments, data broadcast module 337 uses radio 322 for data transmissions. In some embodiments, data broadcast module 337 may format data for transmission (e.g., prepare data packets for transmission). In some embodiments, data broadcast module 337 may transmit packets of data from data streams corresponding to separated data signals generated by data receiving module 331.

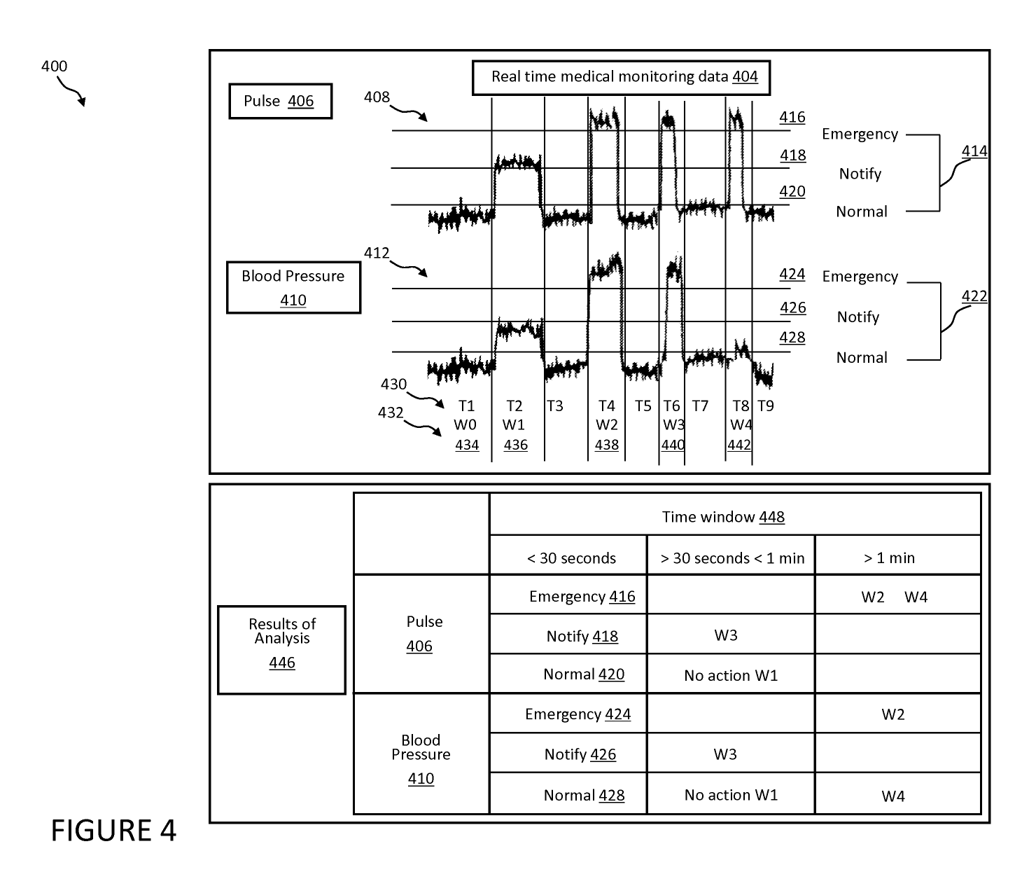

Data analysis module 338 is a software module that analyzes data and reports actions to be taken by user device 120. In some embodiments, data analysis module 338 analyzes real-time physiological data streams received from data receiving module 331 in order to identify emergency and medically significant events. In some embodiments, data analysis module 338 determine whether the real-time physiological data should be stored, or sent to any of a doctor network, third party server, call service based upon data analysis for health issues that may need to alert others. In some embodiments, data analysis module 338 analyzes the data streams received from data receiving module 331 and, based on the analysis, sends commands to one or more of text processor 332, phone processor 333, emergency responder processor 334, and personal assistant processor 335, each of which may send data, messages, or other communications by way of radio 322 and network 365 to one or more of third party server 340, emergency responder 350, and doctor network 360. In some embodiments, data analysis module 338 may generate a command for a particular type of communication based on a determined severity of the identified emergency or medically significant event. For example, data analysis module 338 may identify an event that includes only a minor change in physiological data over a short period of time, determine that the event may not require medical attention, and send a text to user device 320 to continue to monitor the medical situation rather than initiate a 9-1-1 call for emergency help. The data analysis functions performed by data analysis module 338 are described in further detail below in connection with FIGS. 4-6.

Text processor 332 is software module that inputs and outputs text messages and serves as an interface between third party server 140, emergency responder 350 or doctor network 360 on the one hand, and any analyzing data unit on the other hand. Text processor 332 may, upon receiving a command, send one or more standardized text messages via radio 322 to the cloud 130, and from there to third party server 140, emergency responder 350 or doctor network 360. These messages may also contain attached medical monitoring data. Text processer 332 may also receive standardized text messages from one or more of third party server 140, emergency responder 350 or doctor network 360 and pass the command or data contained therein to analyze 338 for additional analysis. Text processor 332 may be, for example, a programmable microchip such as is commonly known in the art.

Phone processor 333 is a programming module that inputs and outputs audio messages and serves as an interface between third party server 140, emergency responder 350 or doctor network 360 on the one hand, and any analyzing data unit on the other hand. Phone processor 333 may, upon receiving a command send one or more standardized audio messages via radio 322 to the cloud 130, and from there to third party server 140, emergency responder 350 or doctor network 360. These messages may also contain attached medical monitoring data in either digital or analog format. These messages may also contain standard telephony signals in order to navigate telephone menu trees, such as is commonly known in the art. Phone processor 333 may also receive audio messages from one or more of third party server 140, emergency responder 350 or doctor network 360. Phone processor 333 may optionally contain a speech-to-text processing software or may process only standardized non-speech messages, such as code groups of symbolic sounds, and pass the command or data contained therein to data analysis module 338 (as described hereinafter) for additional analysis. Phone processor 333 may be, for example, a programmable microchip such as is commonly known in the art.

Emergency responder processor 334 may include any suitable software or processing modules for automatically contacting and interfacing with emergency responder 350. In some embodiments, emergency responder processor 334 transmit medical monitoring data (i.e., physiological data) to medical professionals or first responders associated with emergency responder 350 over network 365.

Personal assistant processor may include any suitable software or processing modules for activating a digital personal assistant running on user device 320. In some embodiments, personal assistant processor 335 provides medical monitoring data and analysis to the personal assistant, which then parses the data and analysis for important information. In some embodiments, the digital personal assistant may perform parsing according to an existing parsing algorithm associated with the personal assistant and user device. In some embodiments, the personal assistant may generate and manage outputting of notifications based on the parsed data to third party server 340, emergency responder 350, doctor network 360, or user device 320. In some embodiments, personal assistant processor generates an audio notification, which is provided directly to a user via speaker 390.

FIG. 4 is an exemplary diagram of a method 400 for generating notifications based on real-time analysis of physiological data on a user device. In the illustrated embodiment, diagram 400 shows data streams of real-time physiological data, which may be, for example blood pressure, pulse, ECG, or any other suitable physiological parameters. In some embodiments, method 400 of FIG. 4 may be implemented on data analysis module 338 of FIG. 3.

Real-time medical monitoring data 404 is shown, including real-time medical monitoring of physiological data received from a medical monitoring device (e.g., medical monitoring device 318 of FIG. 3). In some embodiments, the medical monitoring data 404 may include physiological parameter data measured over a given time interval.

Pulse 406 is readout of pulse rate parameter values as determined based on a physiological signal received from a medical monitoring device (e.g., medical monitoring device 318 of FIG. 3). Pulse readout graph 408 is an exemplary record of a user's pulse rate over time as determined based on a received physiological signal.

Blood pressure 410 is readout of blood pressure parameter values as determined based on a physiological signal received from a medical monitoring device (e.g., medical monitoring device 318 of FIG. 3). Blood pressure readout graph 412 is an exemplary record of a user's blood pressure over time as determined based on a received physiological signal.

Threshold levels 414 and 422 include respective parameter specific thresholds, including emergency thresholds 416 and 424, notify thresholds 418 and 426, and normal thresholds 420 and 428. It will be understood that the illustrated parameters, parameter values and thresholds are provided by way of illustration and not by way of limitation and that any suitable parameters, parameter values, and levels may be used. It will also be understood that processing of physiological signals may be processed at a medical monitoring device and that the data received by the user device and system described herein may be computed physiological parameter values computed in real-time, over time.

Results of analysis table 446 shows results of the analysis of physiological data (e.g., real-time medical monitoring data 404). In the illustrated embodiment, results of analysis table 446 shows pulse 406 and blood pressure 410 and corresponding thresholds for each parameter (i.e., emergency, notify, and normal thresholds) as rows and time windows 448 as columns. Time windows 448 are shown as including a short window (i.e., less than 30 seconds), a moderate window (i.e., between 30 seconds and 1 minute), and a long window (i.e., greater than 1 minute). Results of analysis table 446 is populated with information derived from the analysis of physiological data, as described below.

In some embodiments, the system may analyze pulse rate 406 and blood pressure 410 to determine actions to be taken (i.e., notifications to be delivered). In some embodiments, the actions to be taken may include delivering different types of notifications corresponding to differing severities of respective physiological events, including, for example, emergency, notify, and normal levels of respective physiological values, as shown in results of analysis table 446.

In some embodiments, the system may generate differing types of notifications based on the severity of an identified medical event. In some embodiments, the system generates severity based on, for example, magnitude of parameter values, and duration of a threshold crossing. For example, parameter levels 414 and 422, described above, may correspond to thresholds, each including multiple levels of thresholds particular to the respective physiological parameter, pulse 406 and blood pressure 410. When computed values of a physiological parameter exceed a first threshold, a timer may be started that counts the time the physiological parameter values are above the first threshold and beneath a second threshold. If the physiological parameter values fall below the first threshold, the timer will be stopped. If the physiological parameter values exceed the second threshold, which may correspond to a greater level of severity than the first threshold, then the system may start a second timer. The second timer may count the time the physiological parameter values exceed the second threshold.

In the illustrated embodiment, normal thresholds 428 and 420 correspond to a low/normal severity level, notify thresholds 426 and 418 correspond to an intermediate severity level, and emergency thresholds 416 and 424 correspond to a high severity level. In some embodiments, normal thresholds 428 and 420 correspond to physiological values falling within a range of acceptable values for the particular physiological parameter being measured. In some embodiments, notify thresholds 426 and 418 correspond to physiological parameter values that exceed or fall below a normal level and may require non-immediate medical attention, but that are not yet the level of an emergency. For example, notify thresholds may indicate a need for preventative care in order to prevent a physiological parameter from escalating to emergency level values. In some embodiments, emergency thresholds 416 and 424 correspond to physiological parameter values that fall outside a safe range and require immediate medical attention. For example, the system may trigger a 9-1-1 call when emergency thresholds 416 or 424 are triggered.

It will be understood that the thresholds 414 are described together with thresholds 422 for brevity, but that thresholds 414 are particular to pulse rate parameter values and thresholds 422 are particular to blood pressure parameter values. It will also be understood that any suitable numbers and severity of thresholds may be used, as long as the thresholds are in accordance with the physiological range of the parameter being assessed. In some embodiments, the particular threshold levels may be set by a user, doctor, medical software, medical database, or medical monitoring device. In some embodiments, thresholds 414 and 422 may be minimum parameter value thresholds, which are triggered by the physiological parameter values falling below the respective thresholds. In some embodiments, thresholds 414 and 422 may include both minimum and maximum parameter value thresholds, defining a range of parameter values for each threshold level.

As described above, the system may compute the severity of an event based on the severity of a threshold triggered and the duration of the time period during which the physiological parameter values fall in a particular range defined by the threshold (e.g., above a notify threshold and below an emergency threshold). In the illustrated embodiment, exemplary time windows 430, including T1-T9, are shown beneath pulse rate readout graph 408 and blood pressure readout graph 412. Time windows 430 may be of any suitable duration short enough to capture a medically significant variation in a physiological parameter and long enough to avoid an overabundance of false positives due to oversensitivity. It will be understood that time windows 430 may be computed based on a particular physiological parameter being measured. For example, time windows 430 may be shorter for a physiological parameter that experiences spikes in value only in more severe situations. Time windows 430 may also be of durations specific to a particular patient's physiology (e.g., a patient may have a condition that requires a more or less sensitive time window duration).

The illustrated event widths 432 may correspond to the time duration (i.e., width on the horizontal time axis) of a period during which a monitored physiological parameter (e.g., blood pressure 410 or pulse rate 406) has triggered a threshold (i.e., is above or below a particular threshold without crossing the next severity threshold). In the illustrated embodiment, event widths 432 are shown as including events 434-442, corresponding to respective event widths W0-W4, showing various combinations of threshold crossings and durations of threshold crossings for monitored physiological parameter values (i.e., pulse rate 406 and blood pressure 410). For example, event 440, shown as having width W3, is shown as corresponding to durations of physiological parameter values exceeding emergency threshold levels for both pulse rate 406 with corresponding emergency threshold 416 and blood pressure 410 with corresponding emergency threshold 424. As shown in the results of analysis chart 446, however, the time duration, corresponding to the width W3 of event 440 is falls within a moderate severity duration range (i.e., greater than 30 seconds and less than 1 minute), so the system may determine that event 440 is not an emergency event, but rather a “notify” event of moderate severity for each of the physiological parameters monitored (i.e., pulse rate 450 and blood pressure 452).

In some embodiments, the system may re-categorize an event depending on the duration of the event width. In some embodiments, event widths less than 30 seconds (i.e., short time window) may be disregarded. In some embodiments, event widths between 30 seconds and 1 minute (i.e., moderate time window) may be re-categorized to a lower level, depending on the severity level of the threshold crossed by the physiological monitor being monitored and registering an event. In some embodiments, event widths greater than 1 minute (i.e., long time window) may not be re-categorized as a lower level. In another example, the system is shown to determine that event 442, of event width W4, should give rise to an emergency notification for pulse rate, having exceeded emergency threshold 416 for a long duration of time, but a normal notification for blood pressure, having only exceeded normal threshold 428 for the long period of time.

It will be understood that the time windows 448 shown in results of analysis table 446 may include any suitable time window durations. In some embodiments, time windows 448 may be specific to a particular physiological parameter being monitored or to the physiology of a particular user.

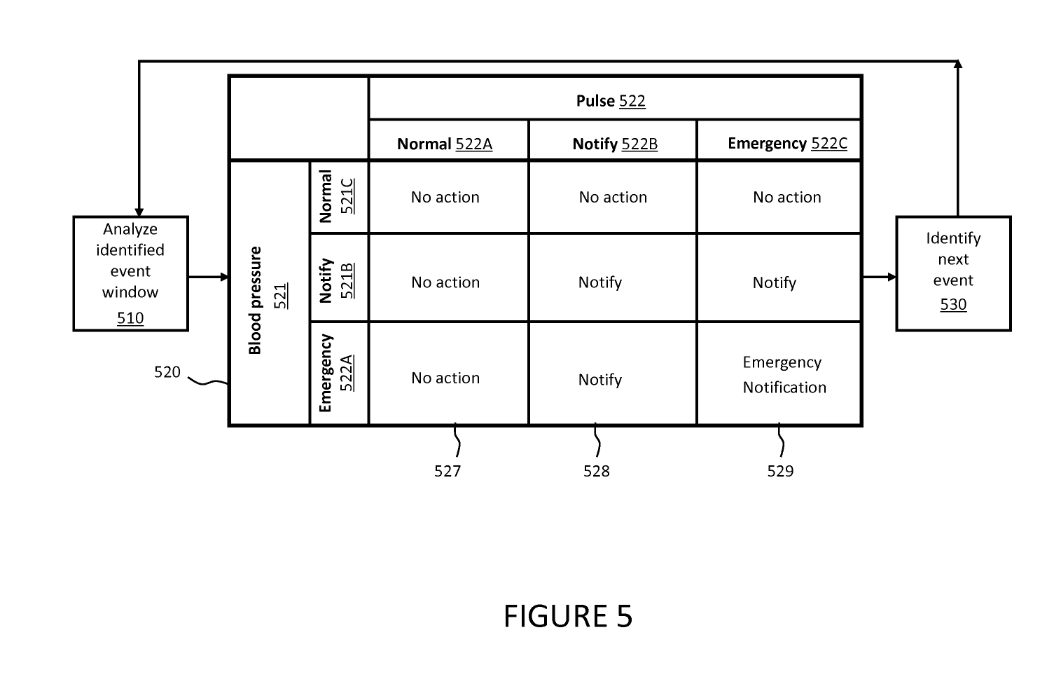

FIG. 5 is an exemplary diagram 500 for analyzing physiological data in real-time on a user device. FIG. 5, as depicted, includes analyze identified event window block 510, logic block 520, and identify next event block 530.

At block 510, the system analyzes physiological data associated with an identified event window. An event may be identified based on real-time physiological data, as discussed above in connection with FIG. 4, and may correspond, for example, to an emergency or a medically significant event. In some embodiments, event data may correspond to data from a particular physiological parameter feed from a particular time window. In some embodiments, an event may correspond to data within an identified event width duration, for example, W0-W4, as described in connection with FIG. 4. In some embodiments, analyzing identified event window block 510 may include transmitting physiological data for the duration of the event width to logic block 520 for further analysis. In the illustrated embodiment, analyzing identified event window block 510 is shown as providing input data for both physiological parameters, the data for each recorded during the same identified event window.

In some embodiments, logic block 520 may take as input physiological event data from block 510 for one or more physiological parameters during an event window previously determined to be associated with at least one medically significant event, as described in FIG. 4. In some embodiments, logic block 520 analyzes the input physiological event data by placing the event data in a correct position in a decision making block based on the analysis of the identified event severities described in FIG. 4. In some embodiments, logic block 520 combines physiological data from more than one physiological parameter measured during the event window to identify an action to be taken. In the illustrated embodiment, logic block 520 is shown as analyzing pulse data 522 and blood pressure data 521. Logic block 520 shows a matrix of actions to be taken for different combinations of event severities for the two physiological parameters measured. Logic block 520 shows pulse rate event classifications normal 522A, notify 522B, and emergency 522C, which may be based on respective pulse rate thresholds 420, 418, and 416 of FIG. 4, and blood pressure event classifications 521A, 521B, 521C, which may be based on respective blood pressure thresholds 428, 426, and 424 of FIG. 4. In some embodiments, event classifications may be based on the results of analysis table 446 of FIG. 4. Logic block 520 may determine, based on the even classifications for both physiological parameters, to take no action 527, notify 528 a specified party (e.g., based on settings), or provide emergency notification 529. Logic block 520 thus shows various actions the system may take when more than one event is identified during an event window.

Identify next event window block 530 may include reading physiological data from another real-time medical monitoring event window, recording the physiological data, and returning to block 510 to re-start the process for determining an action to take.

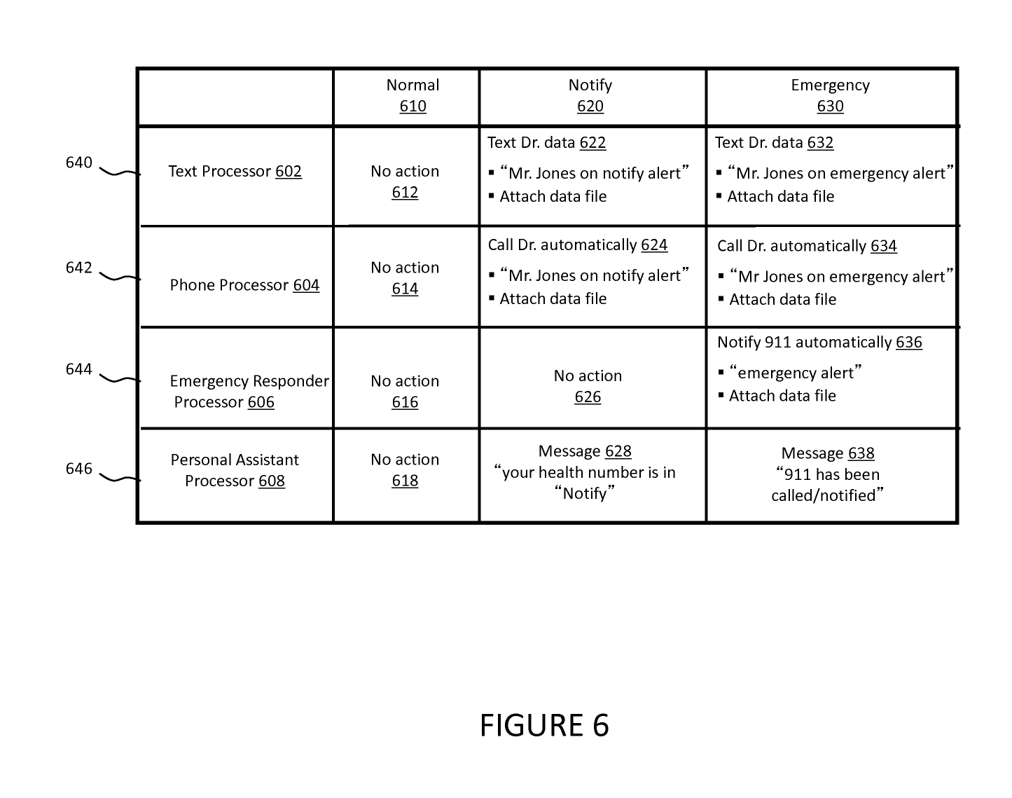

FIG. 6 is an exemplary diagram showing a decision matrix 600 for analyzing physiological data in real-time on a user device. In the illustrated embodiment, decision matrix 600 is shown as including rows corresponding to various processors to deliver notifications, including, text processor 602, phone processor 604, emergency responder processor 606, and personal assistant processor 608. Text Processor 602 may correspond to text processor 332; phone processor 604 may correspond to phone processor 333; emergency response processor 606 may correspond to emergency response processor 334; and personal assistant processor 608 may correspond to personal assistant processor 335, all as described above in connection with FIG. 3. Decision matrix 600 is also shown as including columns corresponding to event classifications normal 610, notify 620, and emergency 630. In some embodiments, event classifications 610, 620, and 630 may be determined based on the severity analysis described in connection with FIG. 4. In some embodiments, event classifications 610, 620, and 630 may be determined using logic block 520 described in connection with FIG. 5.

In row 640, decision matrix 600 shows exemplary actions 612, 622, and 632 that text processor 602 may take when a medical event is respectively classified as normal 610, notify 620, and emergency 630. In row 642, decision matrix 600 shows exemplary actions 614, 624, and 634 that phone processor 604 may take when a medical event is respectively classified as normal 610, notify 620, and emergency 630. In row 644, decision matrix 600 shows exemplary actions 616, 626, and 636 that emergency responder processor 606 may take when a medical event is respectively classified as normal 610, notify 620, and emergency 630. In row 646, decision matrix 600 shows exemplary actions 618, 628, and 638 that personal assistant processor 608 may take when a medical event is respectively classified as normal 610, notify 620, and emergency 630.

As shown, “no action” is taken by each of processors 602-608 when an event is determined to be of a normal 610 classification.

For events the system determines to be of a notify 620 classification, the outputs of the processors 602-608 correspond to various non-emergency notifications to a medical provider corresponding to each of the types of communications of processors 602-608:

Notify text processor action 622 includes texting a user's medical provider with the automatic message: “Mr. Jones on Notify alert” and including the physiological data file as an attachment. Notify phone action 634 includes calling a user's medical provider, which may involve calling a doctor or a doctor network (e.g., doctor network 360 of FIG. 3) with the automatic message: “Mr. Jones on Notify alert” and including the physiological data file as an attachment. Notify emergency responder action 626 is to take no action. Notify personal assistant action 638 involves sending an audio message to a user (e.g., via a speaker of a user device) describing the notify event, for example, “your health number is in ‘Notify’ alert”.

For events the system determines to be of an emergency 630 classification, the outputs of the processors 602-608 correspond to various emergency notifications to a medical provider as well as an emergency responder (e.g., emergency responder 350 of FIG. 3), the notifications corresponding to each of the types of communications of processors 602-608:

Emergency text action 642 includes sending a text message, with optional indicia indicating urgency, to a doctor or a doctor network the automatic message: “Mr. Jones on Emergency alert” and including the physiological data file as an attachment. Emergency phone action 644 includes calling a user's medical provider with the automatic message: “Mr. Jones on Emergency alert” and including a physiological data file as an attachment. Emergency responder action 646 includes contacting the 9-1-1 emergency dispatch system and providing information necessary for first responders to locate a user and render immediate aid, for example, automatically calling 911 with the message: “Emergency alert for health number 9234-765” and including a physiological data file as an attachment. Emergency personal assistant 648 involves sending an audio message to a user (e.g., via speaker of user device) with the automatic message: “911 has been notified/called”.

It will be understood that the above-described exemplary actions, notifications, and processors shown in the illustrated embodiment are merely exemplary and that any suitable actions and notifications may be taken by any suitable processors of software of a user device based on the physiological data analysis.

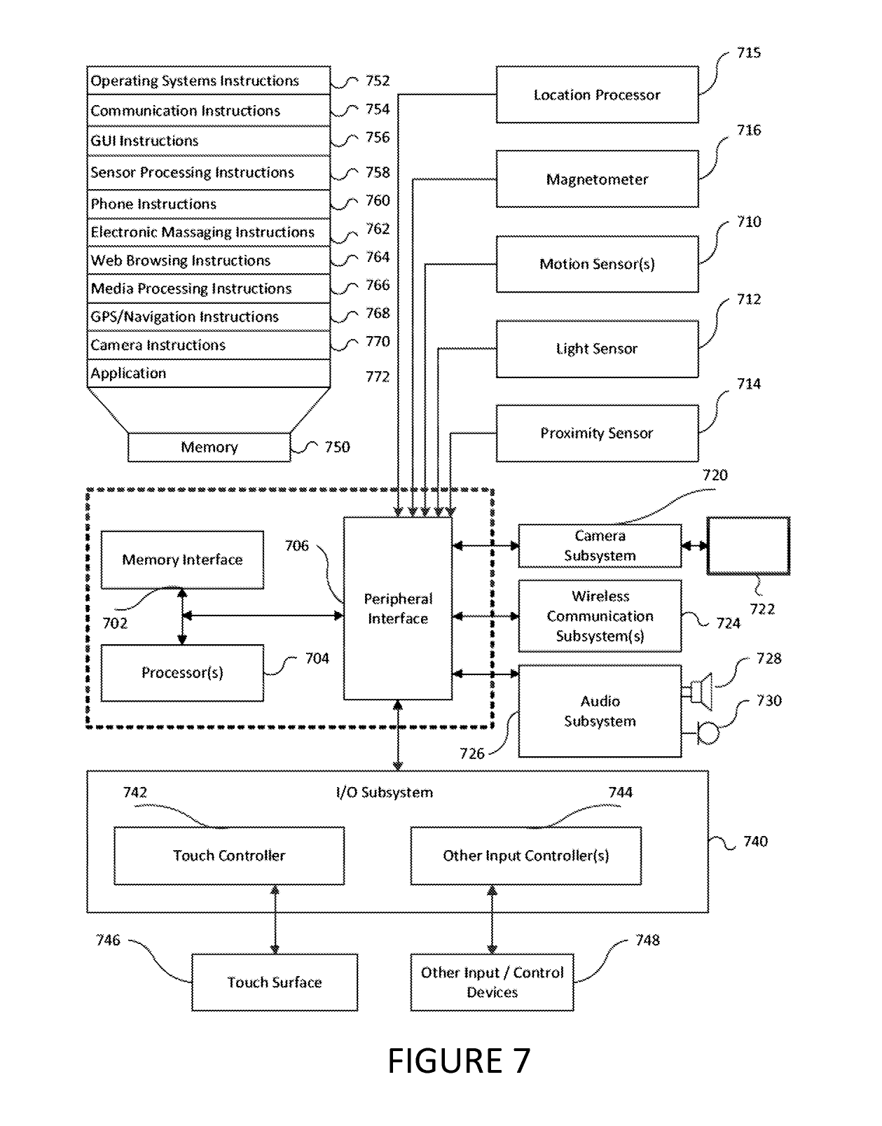

FIG. 7 illustrates a mobile device architecture that may be utilized to implement the various features and processes described herein. Architecture 700 can be implemented in any number of portable devices including but not limited to smart phones, electronic tablets, and gaming devices. Architecture 700 as illustrated in FIG. 7 includes memory interface 702, processors 704, and peripheral interface 706. Memory interface 702, processors 704 and peripherals interface 706 can be separate components or can be integrated as a part of one or more integrated circuits. The various components can be coupled by one or more communication buses or signal lines.

Processors 704 as illustrated in FIG. 7 is meant to be inclusive of data processors, image processors, central processing unit, or any variety of multi-core processing devices. Any variety of sensors, external devices, and external subsystems can be coupled to peripherals interface 706 to facilitate any number of functionalities within the architecture 700 of the exemplar mobile device. For example, motion sensor 710, light sensor 712, and proximity sensor 714 can be coupled to peripherals interface 706 to facilitate orientation, lighting, and proximity functions of the mobile device. For example, light sensor 712 could be utilized to facilitate adjusting the brightness of touch surface 746. Motion sensor 710, which could be exemplified in the context of an accelerometer or gyroscope, could be utilized to detect movement and orientation of the mobile device. Display objects or media could then be presented according to a detected orientation (e.g., portrait or landscape).

Other sensors could be coupled to peripherals interface 706, such as a temperature sensor, a biometric sensor, or other sensing device to facilitate corresponding functionalities. Location processor 715 (e.g., a global positioning transceiver) can be coupled to peripherals interface 706 to allow for generation of geo-location data thereby facilitating geo-positioning. An electronic magnetometer 716 such as an integrated circuit chip could in turn be connected to peripherals interface 706 to provide data related to the direction of true magnetic North whereby the mobile device could enjoy compass or directional functionality. Camera subsystem 720 and an optical sensor 722 such as a charged coupled device (CCD) or a complementary metal-oxide semiconductor (CMOS) optical sensor can facilitate camera functions such as recording photographs and video clips.

Communication functionality can be facilitated through one or more communication subsystems 724, which may include one or more wireless communication subsystems. Wireless communication subsystems 724 can include 802.x or Bluetooth transceivers as well as optical transceivers such as infrared. Wired communication system can include a port device such as a Universal Serial Bus (USB) port or some other wired port connection that can be used to establish a wired coupling to other computing devices such as network access devices, personal computers, printers, displays, or other processing devices capable of receiving or transmitting data. The specific design and implementation of communication subsystem 724 may depend on the communication network or medium over which the device is intended to operate. For example, a device may include wireless communication subsystem designed to operate over a global system for mobile communications (GSM) network, a GPRS network, an enhanced data GSM environment (EDGE) network, 802.x communication networks, code division multiple access (CDMA) networks, or Bluetooth networks. Communication subsystem 724 may include hosting protocols such that the device may be configured as a base station for other wireless devices. Communication subsystems can also allow the device to synchronize with a host device using one or more protocols such as TCP/IP, HTTP, or UDP.

Audio subsystem 726 can be coupled to a speaker 728 and one or more microphones 730 to facilitate voice-enabled functions. These functions might include voice recognition, voice replication, or digital recording. Audio subsystem 726 in conjunction may also encompass traditional telephony functions.

I/O subsystem 740 may include touch controller 742 and/or other input controller(s) 744. Touch controller 742 can be coupled to a touch surface 746. Touch surface 746 and touch controller 742 may detect contact and movement or break thereof using any of a number of touch sensitivity technologies, including but not limited to capacitive, resistive, infrared, or surface acoustic wave technologies. Other proximity sensor arrays or elements for determining one or more points of contact with touch surface 746 may likewise be utilized. In one implementation, touch surface 746 can display virtual or soft buttons and a virtual keyboard, which can be used as an input/output device by the user.

Other input controllers 744 can be coupled to other input/control devices 748 such as one or more buttons, rocker switches, thumb-wheels, infrared ports, USB ports, and/or a pointer device such as a stylus. The one or more buttons (not shown) can include an up/down button for volume control of speaker 728 and/or microphone 730. In some implementations, device 700 can include the functionality of an audio and/or video playback or recording device and may include a pin connector for tethering to other devices.

Memory interface 702 can be coupled to memory 750. Memory 750 can include high-speed random access memory or non-volatile memory such as magnetic disk storage devices, optical storage devices, or flash memory. Memory 750 can store operating system 752, such as Darwin, RTXC, LINUX, UNIX, OS X, ANDROID, WINDOWS, or an embedded operating system such as VxWorks. Operating system 752 may include instructions for handling basic system services and for performing hardware dependent tasks. In some implementations, operating system 752 can include a kernel.

Memory 750 may also store communication instructions 754 to facilitate communicating with other mobile computing devices or servers. Communication instructions 754 can also be used to select an operational mode or communication medium for use by the device based on a geographic location, which could be obtained by the GPS/Navigation instructions 768. Memory 750 may include graphical user interface instructions 756 to facilitate graphic user interface processing such as the generation of an interface; sensor processing instructions 758 to facilitate sensor-related processing and functions; phone instructions 760 to facilitate phone-related processes and functions; electronic messaging instructions 762 to facilitate electronic-messaging related processes and functions; web browsing instructions 764 to facilitate web browsing-related processes and functions; media processing instructions 766 to facilitate media processing-related processes and functions; GPS/Navigation instructions 768 to facilitate GPS and navigation-related processes, camera instructions 770 to facilitate camera-related processes and functions; and instructions 772 for any other application that may be operating on or in conjunction with the mobile computing device. Memory 750 may also store other software instructions for facilitating other processes, features and applications, such as applications related to navigation, social networking, location-based services or map displays.

Each of the above identified instructions and applications can correspond to a set of instructions for performing one or more functions described above. These instructions need not be implemented as separate software programs, procedures, or modules. Memory 750 can include additional or fewer instructions. Furthermore, various functions of the mobile device may be implemented in hardware and/or in software, including in one or more signal processing and/or application specific integrated circuits.

Certain features may be implemented in a computer system that includes a back-end component, such as a data server, that includes a middleware component, such as an application server or an Internet server, or that includes a front-end component, such as a client computer having a graphical user interface or an Internet browser, or any combination of the foregoing. The components of the system can be connected by any form or medium of digital data communication such as a communication network. Some examples of communication networks include LAN, WAN and the computers and networks forming the Internet. The computer system can include clients and servers. A client and server are generally remote from each other and typically interact through a network. The relationship of client and server arises by virtue of computer programs running on the respective computers and having a client-server relationship to each other.

One or more features or steps of the disclosed embodiments may be implemented using an API that can define on or more parameters that are passed between a calling application and other software code such as an operating system, library routine, function that provides a service, that provides data, or that performs an operation or a computation. The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer will employ to access functions supporting the API. In some implementations, an API call can report to an application the capabilities of a device running the application, such as input capability, output capability, processing capability, power capability, and communications capability.

The foregoing detailed description of the technology herein has been presented for purposes of illustration and description. It is not intended to be exhaustive or to limit the technology to the precise form disclosed. Many modifications and variations are possible in light of the above teachings. The described embodiments were chosen in order to best explain the principles of the technology and its practical application to thereby enable others skilled in the art to best utilize the technology in various embodiments and with various modifications as are suited to the particular use contemplated. It is intended that the scope of the technology be defined by the claims appended hereto.

1. A method for analyzing physiological parameter data in real-time on a user device, the method comprising:
receiving user input via a user interface of a user device, wherein the received user input includes medical monitor settings; and
executing instructions stored in memory, wherein the execution of the instructions by the processor:
activates a medical monitor feature on the user device based on the medical monitor settings,
retrieves physiological parameter data in real-time from a physiological monitor associated with a user of the user device based on the medical monitor settings, wherein the physiological parameter data includes values of the physiological parameter of the user measured in real-time,
analyzes, via the user device, the received physiological parameter data in real-time to identify a particular medical event associated with the user, wherein analyzing the received physiological parameter data includes determining that the physiological parameter data includes values that are outside of a predetermined range for the physiological parameter, wherein the particular medical event corresponds to a particular period of time in which the physiological parameter values remain outside the predetermined range, and wherein the particular period of time includes a first period of time corresponding to a short period of time associated with a first medical event that does not require medical attention and a second period of time corresponding to a longer period of time compared to the first period of time associated with a second medical event that does require medical attention, and
generating, via the user device, one or more medical notifications in response to identifying the second medical event, wherein the one or more medical notifications are generated in real-time based on the medical monitor settings.

2. The method of claim 1, wherein the medical monitor settings designate allowed medical monitors, and wherein physiological parameter data is retrieved only from the allowed medical monitors.

3. The method of claim 2, wherein the allowed medical monitors include a blood pressure monitor.

4. The method of claim 2, wherein the allowed medical monitors include a pulse rate monitor.

5. The method of claim 1, wherein the medical monitor settings designate allowed medical service providers, and wherein the one or more medical notifications are delivered only to the allowed medical service providers.

6. The method of claim 5, wherein the allowed medical service providers include a user-specified physician.

7. The method of claim 5, wherein the allowed medical service providers include an emergency response system.

8. The method of claim 1, wherein determining that the physiological parameter data includes values that are outside of a predetermined range for the physiological parameter includes determining that one or more values of the physiological parameter cross a first threshold, wherein the first threshold is a first distance from a maximum value of the predetermined range of the physiological parameter.

9. The method of claim 8, wherein determining that the physiological parameter data includes values that are outside of a predetermined range for the physiological parameter includes determining that one or more values of the physiological parameter meets a second threshold, wherein the second threshold is a second distance from the maximum value of the predetermined range of the physiological parameter, and wherein the second distance is greater than the first distance and the second threshold is greater than the first threshold.

10. The method of claim 9, wherein the execution of instructions by the processor further includes analyzing the identified medical event to determine an associated severity level.

11. The method of claim 10, wherein the one or more medical notifications are generated based on the severity level of the identified medical event.

12. The method of claim 11, wherein the severity level for an identified medical event associated with physiological parameter values that cross the first threshold but not the second threshold is lower than the severity level for an identified medical event associated with physiological parameter values that cross both the first threshold and the second threshold.

13. The method of claim 12, wherein the severity level associated with crossing the first threshold is a non-emergency medical, and wherein the severity level associated with crossing both the first and second thresholds is an emergency medical event.

14. The method of claim 13, wherein the severity level associated with the identified medical event is further based on a duration of the period of time of the medical event, wherein the duration is based on the greater of the first and second thresholds crossed by the physiological parameter values associated with the identified medical event.

15. The method of claim 14, further comprising transmitting the one or more notifications generated based on the identified medical event over a network to a medical service provider when the identified medical event is a non-emergency medical event.

16. The method of claim 14, further comprising transmitting at least one of the one or more notifications generated based on the identified medical event over a network to an emergency response system by the user device when the identified medical event is an emergency medical event.

17. The method of claim 1, wherein the user device is a smartphone.

18. The method of claim 17, wherein activating the medical monitor feature comprises executing a medical monitor application on the smartphone.

19. An apparatus for analyzing physiological parameter data in real-time on a user device, the apparatus comprising:
a user interface that receives user input, wherein the received user input includes medical monitor settings; and
a processor that executes instructions stored in memory to:
activate a medical monitor feature on the user device based on the medical monitor settings,
retrieve physiological parameter data in real-time from a physiological monitor associated with a user of the user device, wherein the physiological parameter data includes values of the physiological parameter of the user measured in real-time, and wherein the physiological parameter data is retrieved based on the medical monitor settings,
analyze the received physiological parameter data in real-time to identify a particular medical event associated with the user, wherein analyzing the received physiological parameter data includes determining that the physiological parameter data includes values that are outside of a predetermined range for the physiological parameter, wherein the particular medical event corresponds to a particular period of time in which the physiological parameter values remain outside the predetermined range, and wherein the particular period of time includes a first period of time corresponding to a short period of time associated with a first medical event that does not require medical attention and a second period of time corresponding to a longer period of time compared to the first period of time associated with a second medical event that does require medical attention, and
generate one or more medical notifications in response to identifying the medical event, wherein the one or more medical notifications are generated in real-time based on the medical monitor settings.

20. A non-transitory computer-readable storage medium, having embodied thereon a program executable by a processor for analyzing physiological parameter data in real-time on a user device, the method comprising:
receiving user input via a user interface of a user device, wherein the received user input includes medical monitor settings;
activating a medical monitor feature on the user device based on the medical monitor settings;
retrieving physiological parameter data in real-time from a physiological monitor associated with a user of the user device, wherein the physiological parameter data includes values of the physiological parameter of the user measured in real-time, and wherein the physiological parameter data is retrieved based on the medical monitor settings;
analyzing, via the user device, the received physiological parameter data in real-time to identify a particular medical event associated with the user, wherein analyzing the received physiological parameter data includes determining that the physiological parameter data includes values that are outside of a predetermined range for the physiological parameter, wherein the particular medical event corresponds to a particular period of time in which the physiological parameter values remain outside the predetermined range, and wherein the particular period of time includes a first period of time corresponding to a short period of time associated with a first medical event that does not require medical attention and a second period of time corresponding to a longer period of time compared to the first period of time associated with a second medical event that does require medical attention; and
generating, via the user device, one or more medical notifications in response to identifying the second medical event, wherein the one or more medical notifications are generated in real-time based on the medical monitor settings.
